(19) United States
(12) Patent Application Publication
(10) Pub. No.: US 2022/0409333 A1
(43) Pub. Date: Dec. 29, 2022

(54) NEW AND IMPROVED DRILL BIT FOR PREPARING A DENTAL IMPLANT SITE FOR THE IMPLANTATION OF A DENTAL IMPLANT

(71) Applicant: A. J. ANTUNES & CO., CAROL STREAM, IL (US)

(72) Inventor: SHLOMO LAZAROVITCH, HERZELIA (IL)

(21) Appl. No.: 17/358,376

(22) Filed: Jun. 25, 2021

(51) Int. Cl. A61C 1/08; A61C 8/00

(52) U.S. Cl. CPC A61C 1/08; A61C 8/0089

(57) ABSTRACT

A new and improved drill bit for preparing a dental implant site within a jawbone for the implantation of a dental implant, as a result of being rotatably driven by a motor drive, comprises a body portion which includes an upper shank portion to be inserted into the motor drive, and a lower drilling portion for forming a hole or bore with a jawbone within which a dental implant is to be implanted. The lower drilling portion includes a pointed conical tip portion having an apex for determining the location of the implantation site, and the outer diametrical extent of the lower drilling portion progressively increases in diameter as considered in the direction from the apex to the upper shank portion so as to automatically enlarge the hole or bore formed within the jawbone such that the complete drilling process can be completed within a single drilling operation as opposed to conventional operations which require the use of a plurality of drill bits of increasingly larger diametrical extents.

### FIELD OF THE INVENTION

The present invention relates generally to tools, and more particularly to a drill bit which is to be utilized in connection with the preparation of a dental implant site into which a dental implant is to be implanted.

### BACKGROUND OF THE INVENTION

Dental implants are of course well known, as are drills for drilling holes into a jawbone at which location the dental implant is to be implanted. One major drawback with conventional dental implantation methods or procedures resides in the fact that in forming the hole into which the dental implant is to be implanted, the dental surgeon utilizes a plurality of drill bits of progressively increasing diameter so as to progressively increase the diametrical size of the drilled hole in predetermined increments in order to finally accommodate the dental implant. As may therefore be appreciated, this procedure requires an inordinate amount of time because the dental surgeon must first select a first drill bit having a first predetermined diameter, inserting the first drill bit into the drilling tool, drill down to the depth required to accommodate the dental implant, remove the first drill bit from the implant site, remove the first drill bit from the drilling tool, select a second drill bit having a predetermined diameter which is slightly larger than that of the first drill bit, inserting the second drill bit into the drilling tool, drill down to the depth required to accommodate the dental implant so as to widen the diametrical extent of the drilled hole by means of the difference between the diametrical extents of the first and second drill bits, remove the second drill bit from the implant site, and remove the second drill bit from the drilling tool. This procedure is often repeated with a third drill bit which has a diameter which is greater than the diameter of the second drill bit by means of a predetermined amount, as well as a fourth drill bit which has a diameter which is greater than the diameter of the third drill bit by means of a predetermined amount, until the hole has a finalized diameter that can in fact accommodate the dental implant. It can therefore be readily appreciated that this entire procedure is time-consuming and tedious. In addition, such procedure becomes even more time-consuming and tedious when it is realized that the procedure must be repeated for each dental implant to be implanted.

A need therefore exists in the art for a new and improved drill bit which is to be utilized in connection with the preparation of a dental implant site into which a dental implant is to be implanted. An additional need exists in the art for a new and improved drill bit which is to be utilized in connection with the preparation of a dental implant site into which a dental implant is to be implanted and which will effectively eliminate the conventional time-consuming and tedious operations which are currently being experienced in connection with the preparation of a dental implant site. A still additional need exists in the art for a new and improved drill bit which is to be utilized in connection with the preparation of a dental implant site into which a dental implant is to be implanted, which will effectively eliminate the conventional time-consuming and tedious operations which are currently being experienced in connection with the preparation of a dental implant site, and which enables the dental surgeon to quickly prepare the dental implant site for the implantation of a dental implant. A further need exists in the art for a new and improved drill bit which is to be utilized in connection with the preparation of a dental implant site into which a dental implant is to be implanted, which will effectively eliminate the conventional time-consuming and tedious operations which are currently being experienced in connection with the preparation of a dental implant site, and which enables the dental surgeon to quickly prepare the dental implant site for the implantation of a dental implant as a result of effectively rendering the utilization of a multiplicity of different drill bits, having progressively larger diametrical extents, unnecessary.

A still further need exists in the art for a new and improved drill bit which is to be utilized in connection with the preparation of a dental implant site into which a dental implant is to be implanted, which will effectively eliminate the conventional time-consuming and tedious operations which are currently being experienced in connection with the preparation of a dental implant site, which enables the dental surgeon to quickly prepare the dental implant site for the implantation of a dental implant as a result of effectively rendering the utilization of a multiplicity of different drill bits, having progressively larger diametrical extents, unnecessary, and which will enable the dental surgeon to accomplish the necessary drilling of the implantation site with a single drill bit. A yet further need exists in the art for a new and improved drill bit which is to be utilized in connection with the preparation of a dental implant site into which a dental implant is to be implanted, which will effectively eliminate the conventional time-consuming and tedious operations which are currently being experienced in connection with the preparation of a dental implant site, which enables the dental surgeon to quickly prepare the dental implant site for the implantation of a dental implant as a result of effectively rendering the utilization of a multiplicity of different drill bits, having progressively larger diametrical extents, unnecessary, and which will enable the dental surgeon to accomplish and complete the entire drilling of the implantation site during a single drilling procedure utilizing a single drill bit.

### Overall Objectives of the Present Invention

An overall objective of the present invention is to provide a new and improved drill bit which is to be utilized in connection with the preparation of a dental implant site into which a dental implant is to be implanted. An additional overall objective of the present invention is to provide a new and improved drill bit which is to be utilized in connection with the preparation of a dental implant site into which a dental implant is to be implanted and which will effectively eliminate the conventional time-consuming and tedious operations which are currently being experienced in connection with the preparation of a dental implant site. A still additional overall objective of the present invention is to provide a new and improved drill bit which is to be utilized in connection with the preparation of a dental implant site into which a dental implant is to be implanted, which will effectively eliminate the conventional time-consuming and tedious operations which are currently being experienced in connection with the preparation of a dental implant site, and which enables the dental surgeon to quickly prepare the dental implant site for the implantation of a dental implant. A further overall objective of the present invention is to provide a new and improved drill bit which is to be utilized in connection with the preparation of a dental implant site into which a dental implant is to be implanted, which will effectively eliminate the conventional time-consuming and tedious operations which are currently being experienced in connection with the preparation of a dental implant site, and which enables the dental surgeon to quickly prepare the dental implant site for the implantation of a dental implant as a result of effectively rendering the utilization of a multiplicity of different drill bits, having progressively larger diametrical extents, unnecessary. A still further overall objective of the present invention is to provide a new and improved drill bit which is to be utilized in connection with the preparation of a dental implant site into which a dental implant is to be implanted, which will effectively eliminate the conventional time-consuming and tedious operations which are currently being experienced in connection with the preparation of a dental implant site, which enables the dental surgeon to quickly prepare the dental implant site for the implantation of a dental implant as a result of effectively rendering the utilization of a multiplicity of different drill bits, having progressively larger diametrical extents, unnecessary, and which will enable the dental surgeon to accomplish the necessary drilling of the implantation site with a single drill bit. A yet further overall objective of the present invention is to provide a new and improved drill bit which is to be utilized in connection with the preparation of a dental implant site into which a dental implant is to be implanted, which will effectively eliminate the conventional time-consuming and tedious operations which are currently being experienced in connection with the preparation of a dental implant site, which enables the dental surgeon to quickly prepare the dental implant site for the implantation of a dental implant as a result of effectively rendering the utilization of a multiplicity of different drill bits, having progressively larger diametrical extents, unnecessary, and which will enable the dental surgeon to accomplish and complete the entire drilling of the implantation site during a single drilling procedure utilizing a single drill bit.

### SUMMARY OF THE INVENTION

The foregoing and other objectives are achieved in accordance with the principles and teachings of the present invention wherein a new and improved drill bit comprises a pointed conical drill tip which effectively serves as a pilot point marking the location at which the drill bit will initiate drilling into a jawbone site where a dental implant is to be implanted. The pointed conical drill tip extends a predetermined axial extent away from a distal apex of the pointed conical drill tip, and side surface portions of the pointed conical drill tip subtend an angle of thirty degrees (30°) with respect to the longitudinal axis of the drill bit. The body of the drill bit extends axially away from the apex of the pointed conical drill tip at an angle of approximately four degrees (4°) with respect to the longitudinal axis of the drill bit so that it can be readily appreciated that as one's position moves axially away from the pointed conical drill tip toward the shank of the drill bit, which will be inserted into a suitable motor drive for driving or rotating the drill bit, the diametrical extent of the drill bit will become predeterminedly larger in a predetermined incremental and linear manner. This is important because as the drill bit enters the implantation site, the size of the drilled hole is automatically diametrically enlarged to the extent required to accommodate a dental implant once the drill bit has been inserted to a predetermined length or depth into the implantation site, whereby there is no need to utilize a plurality of drill bits of progressively larger diametrical extents as has been conventionally done. A stopper is interposed between the drill bit and the shank so as to in fact predetermine the aforenoted length or depth to which the hole in the jawbone is to be drilled so as to accommodate the dental implant.

It is further noted that the drill bit comprises four, longitudinally extending channels upon one side of which are formed cutting blades, wherein the four longitudinally extending channels and cutting blades or edges are formed upon four equiangularly spaced regions of the drill bit wherein the blades extend, in a slightly spiral configuration, from the drill tip or apex up to the stopper. The four blades serve to cut the jawbone as well as to collect jawbone debris and/or marrow which is expelled from the jawbone through the channels. Immediately adjacent to each one of the four equiangularly spaced, slightly spiraled cutting blades or edges, there is also provided four sets of longitudinally extending threads which are respectively interposed between each one of the four longitudinally extending channels and blades and are adapted to form or tap threads within or upon the inner peripheral surface portions of the hole of the jawbone wherein the threads are effectively adapted to match with and threadedly engage the threads of the dental implant when the dental implant is threadedly inserted into the tapped bore defined within the jawbone. The drill bit may be operated at a rotational speed of anywhere between 50-40,000 RPM, and preferably within the range of 800-1000 RPM. The drill bit is operatively connected to a suitable motor drive by means of a suitable reduction gearing whereby, for example, the gearing may selectively comprise a 1:1 drive ratio, or for example, a 1:20 drive ratio.

### BRIEF DESCRIPTIONS OF THE DRAWINGS

Various other features and attendant advantages of the present invention will be more fully appreciated from the following detailed description when considered in connection with the accompanying drawings in which like reference characters designate like or corresponding parts throughout the several views, and wherein:

FIG. 1 is a schematic front elevational view of a new and improved drill bit developed in accordance with the principles and teachings of the present invention;

FIG. 2 is an enlarged cross-sectional view of the pointed conical tip portion of the new and improved drill bit disclosed within FIG. 1;

FIG. 3 is an enlarged view of a portion of the drill bit body clearly showing the details of the cutting blades and the tapping threads; and

FIG. 4 is a schematic view showing the new and improved drill bit of the present invention as it has formed a borehole within a jawbone so as to define a dental implant implantation site and as it is being withdrawn from the formed borehole, portions of the jawbone having been removed and effectively expelled from the borehole.

### DETAILED DESCRIPTION OF THE PREFERRED EMBODIMENT

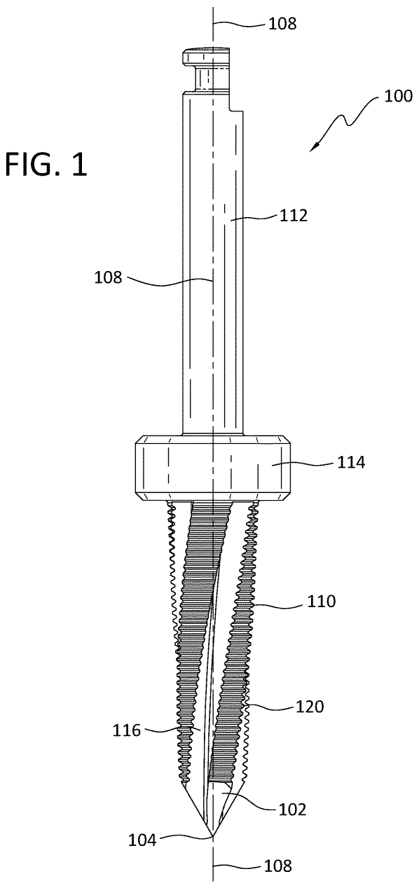

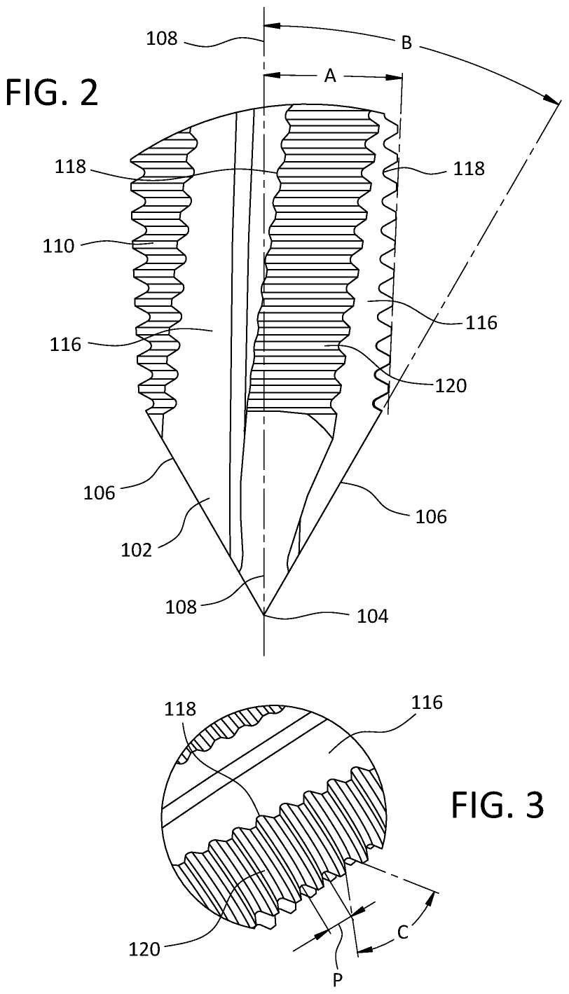

Referring now to the drawings, and more particularly to FIGS. 1-3 thereof, there is disclosed a new and improved drill bit which has been developed in accordance with the principles and teachings of the present invention and which has been designated 100. More particularly, it is seen that the new and improved drill bit 100 is seen to comprise a lower drilling portion which comprises a pointed conical drill tip 102 that has an apex portion 104 which effectively serves as a pilot point marking the location at which the drill bit 100 will initiate drilling into a jawbone site where a dental implant is to be implanted. The pointed conical drill tip 102 extends a predetermined axial extent away from the distal apex portion 104 of the pointed conical drill tip 102, and side surface portions of the pointed conical drill tip 102, which can best be seen and appreciated from FIG. 2 as at 106, subtend an angle B of approximately thirty degrees (30°) with respect to the longitudinal axis 108 of the drill bit 100. The body 110 of the drill bit 100 extends axially away from the apex 104 of the pointed conical drill tip 102 of the drill bit 100 at an angle A of approximately four degrees (4°) with respect to the longitudinal axis 108 of the drill bit 100, as can best be seen in FIG. 2, so that it can be readily appreciated that as one's position moves axially away from the pointed conical drill tip 102 toward an upper shank portion 112 of the drill bit 100, which is adapted to be inserted into a suitable motor drive, not shown, for driving or rotating the drill bit 100, the diametrical extent of the drill bit 100 will become predeterminedly larger in a predetermined incremental and linear manner. This is important because as the drill bit 100 enters the implantation site, the size of the drilled hole is automatically diametrically enlarged to the extent required to accommodate a dental implant once the drill bit 100 has been inserted to a predetermined length or depth into the implantation site, whereby there is no need to utilize a plurality of drill bits of progressively larger diametrical extents as has been conventionally done. A stopper 114 is interposed between the lower drilling portion of the drill bit 100 and the upper shank portion 112 of the drill bit 100 so as to in fact predetermine the aforenoted length or depth to which the hole in the jawbone is to be drilled so as to accommodate the dental implant.

It is further noted that the lower drilling portion of the drill bit 100 comprises four, longitudinally extending channels 116 upon one side of which are formed cutting blades 118, wherein the four longitudinally extending channels 116 and cutting blades or edges 118 are formed upon four equiangularly spaced regions of the lower drilling portion of the drill bit 100 wherein the blades or edges 118 extend, in a slightly spiral configuration, from the drill tip or apex 104 up to the stopper 114. The four blades 118 serve to cut the jawbone as well as to collect jawbone debris and/or marrow which is expelled from the jawbone through the channels 116. Immediately adjacent to each one of the four equiangularly spaced, slightly spiraled cutting blades or edges 118, there is also provided four sets of longitudinally extending threads 120 which are respectively interposed between each one of the four longitudinally extending channels and blades 118 and are adapted to form or tap threads within or upon the inner peripheral surface portions of the hole of the jawbone wherein the threads are effectively adapted to match with and threadedly engage the threads of the dental implant when the dental implant is threadedly inserted into the tapped bore defined within the jawbone.

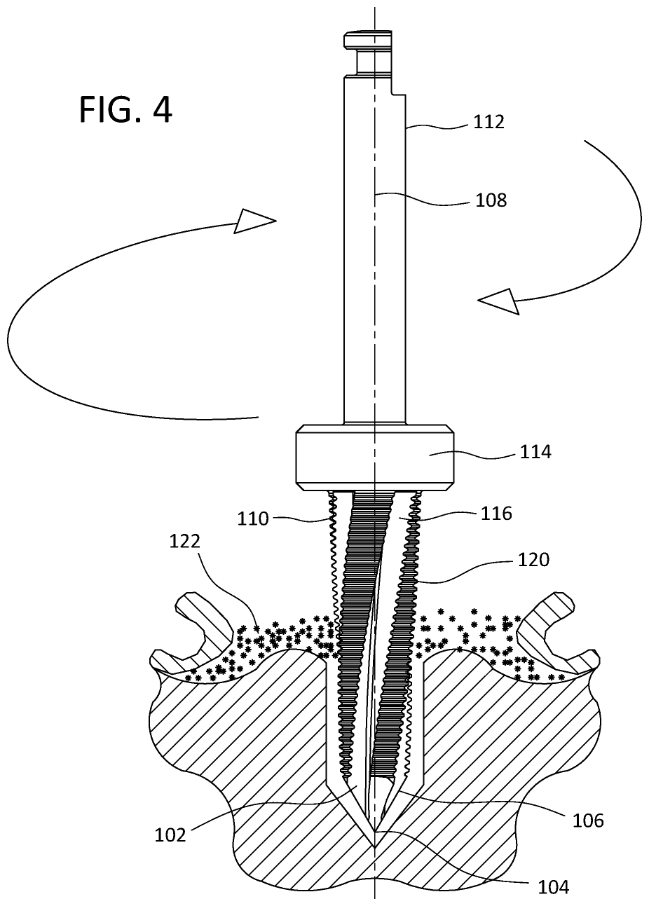

The drill bit 100 may be operated at a rotational speed of anywhere between 50-40,000 RPM, but preferably within the range of 800-1000 RPM. The drill bit 100 is operatively connected to a suitable motor drive, not shown, by means of a suitable reduction gearing, also not shown, whereby, for example, the gearing may selectively comprise a 1:1 drive ratio, or for example, a 1:20 drive ratio. It is also to be noted, as can best be appreciated from FIG. 3 that adjacent threads subtend an angle C of approximately sixty degrees (60°) while the pitch P defined between adjacent threads is approximately 0.3 millimeters. Lastly, it can be appreciated from FIG. 4, that as the drill bit 100 forms the bore within the jawbone, debris 122 from the drilling and boring operation is expelled from the dental implant site through means of the plurality of spiraled channels 116.

Obviously, many variations and modifications of the present invention are possible in light of the above teachings. It is therefore to be understood that within the scope of the appended claims, the present invention may be practiced otherwise than as specifically described herein.

What is claimed as new and desired to be protected by Letters Patent, is:

1. A drill bit for preparing a dental implant site within a jawbone for the implantation of a dental implant as a result of being rotatably driven by a motor drive, comprising:
a body portion defined around a longitudinal axis and comprising an upper shank portion for operative connection to a motor drive, and a lower drilling portion for drilling a hole within a jawbone;
said lower drilling portion a pointed tip having an apex portion formed thereon for defining an insertion point at which a dental implant site is to be formed and into which a dental implant is to be implanted;
a plurality of longitudinally extending channels equidistantly defined around said lower drilling portion;
a plurality of cutting blades formed adjacent to said plurality of longitudinally extending channels for cutting a hole within said dental implant site;
a plurality of sets of threads formed upon said lower drilling portion and disposed immediately adjacent to said cutting blades for tapping threads into the jawbone so as to match and accommodate a threaded portion of the dental implant;
wherein the diametrical extent of said lower drilling portion becomes increasingly larger, as considered in the direction extending from said apex portion toward said upper shank portion, as said drill bit is driven into threaded hole formed within the jawbone by the motor drive, such that the threaded hole defined within the jawbone is completed within one drilling operation as opposed to being formed by a plurality of drill bits of progressively larger diametrical extents.

2. The drill bit as set forth in claim 1, wherein:
said plurality of blades comprise four blades.

3. The drill bit as set forth in claim 2, wherein:
said plurality of sets of threads comprises four sets of threads.

4. The drill bit as set forth in claim 3, wherein:
said plurality of blades and said plurality of sets of threads are alternatively disposed upon said lower drilling portion of said body portion of said drill bit in a circumferentially extending array around said longitudinal axis of said drill bit such that each set of said plurality of sets of threads is interposed between successive blades of said plurality of blades.

5. The drill bit as set forth in claim 3, wherein:
said plurality of blades and said plurality of sets of threads are alternatively disposed upon said lower drilling portion of said body portion of said drill bit in a circumferentially extending array around said longitudinal axis of said drill bit and extend longitudinally in a slightly spiral manner.

6. The drill bit as set forth in claim 3, wherein:
said plurality of blades and said plurality of sets of threads are alternatively disposed upon said lower drilling portion of said body portion of said drill bit in a circumferentially extending array around said longitudinal axis of said drill bit and wherein said plurality of blades and said plurality of sets of threads are equiangularly spaced from each other within said circumferentially extending array.

7. The drill bit as set forth in claim 1, further comprising:
a stopper fixedly mounted upon said body portion between said upper shank portion and said lower drilling portion so as to precisely define the depth to which said drill bit can be inserted into the bone so as to form the implant site into which the dental implant is to be implanted.

8. The drill bit as set forth in claim 1, wherein:
said drill bit is adapted to be operatively connected to a motor drive with an operational ratio of 1:20.

9. The drill bit as set forth in claim 8, wherein:
said drill bit is adapted to be operatively connected to a motor drive with an operational ratio of between 1:20 such that said drill bit can be rotated at a speed of between 50 and 40,000 RPM.

10. The drill bit as set forth in claim 9, wherein:
said drill bit is adapted to be operatively connected to a motor drive with an operational ratio of between 1:20 such that said drill bit can preferably be rotated at a speed of between 800 and 1,000 RPM.

11. The drill bit as set forth in claim 1, wherein:
adjacent threads of said plurality of sets of threads subtend angles of approximately sixty degrees (60); and
adjacent threads of said plurality of sets of threads are spaced apart by approximately 0.3 millimeters such that the pitch of the threads is 0.3 millimeters.

12. The drill bit as set forth in claim 1, wherein:
said plurality of longitudinally extending channels serve as pathways for debris to be expelled from the hole drilled within the jawbone.
